(19) United States
(12) Patent Application Publication
(10) Pub. No.: US 2026/0272299 A1
(43) Pub. Date: Sep. 17, 2026

(54) IMPLANTABLE MEDICAL DEVICE CONFIGURED TO SWITCH FROM AN ACTIVE DEVICE TO A PASSIVE DEVICE

(71) Applicant: Medtronic, Inc., Minneapolis, MN (US)

(72) Inventor: Markus W. Reiterer, Plymouth, MN (US)

(21) Appl. No.: 19/559,022

(22) Filed: Mar. 6, 2026

Related U.S. Application Data
(60) Provisional application No. 63/773,079, filed on Mar. 17, 2025.

(51) Int. Cl. A61B 5/00

(52) U.S. Cl. CPC A61B 5/0031; A61B 5/686; A61B 2560/0209; A61B 2560/0219

(57) ABSTRACT

An implantable medical device (IMD) comprises: a battery; sensing circuitry; communication circuitry configured to communicate via a first type of communication and a second type of communication, the first type of communication being facilitated by the battery and the second type of communication being facilitated via an external power source; and processing circuitry configured to: control the communication circuitry to communicate patient data sensed via the sensing circuitry using the first type of communication when a power level of the battery exceeds a threshold power level; control the communication circuitry to permanently switch from the first type of communication to the second type of communication when the power level of the battery falls below the threshold power level; and control the communication circuitry to communicate patient data sensed in response to receiving power from the external power source.

### RELATED APPLICATIONS

This application claims the benefit of U.S. Provisional Application Serial Number 63/773,079, filed Mar. 17, 2025, the entire contents of which are incorporated herein by reference.

### FIELD

This disclosure generally relates to power management in electronic devices and more particularly, but not exclusively, to implantable monitoring devices.

### BACKGROUND

A variety of implantable medical devices (IMD) have been clinically implanted or proposed for therapeutically treating and/or monitoring cardiac, neurological, and/or other conditions of a patient. IMDs may monitor physiological signals of the patient. In general, using these signals, such devices facilitate monitoring and evaluating patient health over a number of months or years, outside of a clinic setting.

In some cases, such devices may process and/or transmit device data to evaluate patient health. Some devices may receive power for sensing and/or transmitting device data from an internal power source, e.g., a battery of the device, and operate as active devices. Other devices may receive power from an external power source, e.g., an external device configured for proximate power transmission.

### SUMMARY

In general, this disclosure describes techniques for monitoring patients using an implantable medical device (IMD) that operates as an active device during a first period of time and permanently switches to operating as a passive device during a second period of time subsequent to the first period of time. The first period of time may correspond to a battery life of a battery of the IMD. As used herein, active device operation describes device operations in which the device, e.g., the IMD, receives power for performing one or more of communication, sensing, or processing from an internal power source, e.g., a battery. Passive device operation describes operations in which the IMD receives power for performing one or more of communication, sensing, or processing from an external power source, e.g., an external user device.

In some examples, the IMD may be configured to monitor a patient condition. The IMD may, during the first period of time, e.g., weeks, months, or years, communicate via a first type of communication, e.g., Bluetooth® Low Energy (BLE). The IMD may sense patient data, e.g., one or more physiological signals of the patient, via sensing circuitry indicative of the patient condition according to a periodic schedule. The battery of the IMD may supply power to the communication circuitry including a first communication component, e.g., a first antenna. The battery of the IMD may additionally supply power to the sensing circuitry. The battery of the IMD may comprise a primary cell battery. The battery may expire over the course of the first period of time.

During the second period of time, e.g., a period of time subsequent to the expiration of the battery and lasting months or years, the IMD may communicate via a second type of communication, e.g., radio frequency identification (RFID) communication, such as near-field communication (NFC) using a second communication component, e.g., a second antenna. An external power source, e.g., an external user device configured to NFC, may provide power to communication circuitry of the IMD including the second component to facilitate the communication via the second type of communication. The external power source may provide power to sensing circuitry of the IMD to facilitate sensing of patient data. During the second period of time, the IMD may only sense patient data when receiving power from the external power source. The IMD may be configured to sense patient data in response to receiving power from the external power source.

In one example, an IMD includes: a battery; sensing circuitry; communication circuitry configured to communicate via a first type of communication and a second type of communication, the first type of communication being facilitated by the battery and the second type of communication being facilitated via an external power source; and processing circuitry configured to: control the communication circuitry to communicate patient data sensed via the sensing circuitry using the first type of communication when a power level of the battery exceeds a threshold power level; control the communication circuitry to permanently switch from the first type of communication to the second type of communication when the power level of the battery falls below the threshold power level; and control the communication circuitry to communicate patient data sensed in response to receiving power from the external power source.

In another example, a method includes: controlling, by processing circuitry of an implantable medical device (IMD), communication circuitry of the IMD to communicate patient data sensed via sensing circuitry of the IMD using a first type of communication when a power level of a battery of the IMD exceeds a threshold power level; controlling, by the processing circuitry, the communication circuitry to permanently switch from the first type of communication to a second type of communication when the power level of the battery falls below the threshold power level; and controlling, by the processing circuitry, the communication circuitry to communicate patient data sensed in response to receiving power from the external power source.

In another example, a non-transitory computer-readable medium stores instructions that when executed cause processing circuitry to: control communication circuitry of an implantable medical device (IMD) to communicate patient data sensed via sensing circuitry of the IMD using a first type of communication when a power level of a battery of the IMD exceeds a threshold power level; control the communication circuitry to permanently switch from the first type of communication to a second type of communication when the power level of the battery falls below the threshold power level; and control the communication circuitry to communicate patient data sensed in response to receiving power from the external power source.

This summary is intended to provide an overview of the subject matter described in this disclosure. It is not intended to provide an exclusive or exhaustive explanation of the apparatus and methods described in detail within the accompanying drawings and description below. Further details of one or more examples are set forth in the accompanying drawings and the description below.

### BRIEF DESCRIPTION OF DRAWINGS

FIG. 1 illustrates the environment of an example medical device system in conjunction with a patient, in accordance with one or more techniques of this disclosure.

FIGS. 2A and 2B are conceptual diagrams illustrating example implantable medical devices that operate in accordance with one or more techniques of this disclosure.

FIG. 3 is a block diagram illustrating an example configuration of an implantable medical device that operates in accordance with one or more techniques of this disclosure.

FIG. 4 is a block diagram illustrating an example configuration of an external device that operates in accordance with one or more techniques of this disclosure.

FIG. 5 is a flow diagram illustrating an example operation for permanently switching from communicating using a first type of communication to communicating using a second type of communication, in accordance with one or more techniques of this disclosure.

FIG. 6 is a flow diagram illustrating an example operation for permanently switching from sensing according to a periodic schedule via the battery to sensing in response to receiving power from an external power source, in accordance with one or more techniques of this disclosure.

Like reference characters refer to like elements throughout the figures and description.

### DETAILED DESCRIPTION

A variety of types of implantable and external devices are configured to monitor health based on sensed patient data. External devices that may be used to non-invasively sense and monitor ECGs and other physiological signals include wearable devices with electrodes configured to contact the skin of the patient, such as patches, watches, rings, necklaces, hearing aids, a wearable cardiac monitor or automated external defibrillator (AED), clothing, car seats, or bed linens. Such external devices may facilitate relatively longer-term monitoring of patient health during normal daily activities.

Implantable medical devices (IMDs) also sense and monitor patient data indicative of patient conditions. Example IMDs include pacemakers and implantable cardioverter-defibrillators, which may be coupled to intravascular or extravascular leads, as well as pacemakers with housings configured for implantation within the heart, which may be leadless. Some IMDs do not provide therapy, such as implantable patient monitors. One example of such an IMD is the Reveal LINQ™ or LINQ II™ insertable cardiac monitors (ICMs), available from Medtronic, Inc., which may be inserted subcutaneously. Such IMDs may facilitate relatively longer-term continuous monitoring of patients during normal daily activities, and may periodically or on demand transmit collected data, e.g., episode data for detected arrhythmia episodes, data indicative of blood oxygenation, data indicative of impedance, data indicative of temperature, data indicative of posture, data indicative of activity level, and/or chemical sensor data, to a remote patient monitoring system, such as the Medtronic CareLink™ Network via a home monitoring system or a smart phone application.

For ease of implant, it may be desirable for an IMD to be as small as possible. As an example, it may be desirable for a monitoring device to be an injectable IMD. To be injectable, the IMD may need to be sized such that injection of the IMD does not require a stitch at an injection site. In some examples, a size range for an injectable IMD may include the IMD having a diameter or width of up to 2.0 millimeters or 2.2 millimeters. Injectable IMDs may include either rechargeable batteries or small primary cell batteries, with primary cell batteries often being preferred due to primary cell batteries having a higher energy density than rechargeable batteries. These injectable IMDs may have decreased longevity relative to larger devices. Thus, after a relatively short period of time, e.g., 3 months to 18 months, injectable IMDs are no longer useful and must be removed and/or replaced or remain dormant in the body. In some examples, an IMD that might otherwise be sized such that it is injectable may be made larger to accommodate a battery with enough power to operate the IMD for a desired period of time. Although described primarily in the context of an injectable IMD, the techniques of this disclosure may be implemented in any IMD. Likewise, the IMD may be implanted using techniques other than injection.

The devices, systems, and techniques of this disclosure include an IMD configured to use a primary power source, e.g., a primary cell battery or another internal power source, to operate as an active monitoring device for a patient for a first period of time and then switch to operating as a passive monitoring device, e.g., a device that performs sensing and/or communication when powered by an external, proximate source, for a second period of time once the primary cell battery life has ended. In some embodiments, this switch may be permanent or otherwise apply to the remaining life of the device. In other embodiments, the switch may be reversible such as, for example, if the internal power source were replaced, recharged, or otherwise made available to the device again.For example, the IMD may, during the first period of time, e.g., days, weeks, or months, communicate via a first type of communication, e.g., Bluetooth® Low Energy (BLE). The IMD may sense patient data, e.g., one or more physiological signals of the patient, via sensing circuitry indicative of the patient condition. The IMD may sense patient data continuously and/or according to a periodic schedule, e.g., a daily schedule. The battery of the IMD may supply power to the communication circuitry including the first communication component. The battery of the IMD may additionally supply power to the sensing circuitry. The battery of the IMD may comprise a primary cell battery. The battery may expire over the course of the first period of time, e.g., reach the end of its ability to usefully or reliably power operation of the IMD at the end of the first period of time.

During the second period of time, e.g., a period of time subsequent to the expiration of the battery and lasting months or years, the IMD may communicate via a second type of communication, e.g., radio frequency identification (RFID) communication, such as inductive coupling. In some examples, inductive coupling may comprise near-field communication (NFC). An external power source, e.g., an external user device configured to NFC, may provide power to communication circuitry of the IMD to facilitate the communication via the second type of communication. The external power source may provide power to sensing circuitry of the IMD to facilitate sensing of patient data. During the second period of time, the IMD may only sense patient data when receiving power from the external power source. The IMD may be configured to sense patient data in response to receiving power from the external power source.

In some examples, the IMD may be configured to monitor a patient condition related to a procedure, e.g., a surgical procedure, and/or a health event, e.g., a myocardial infarction, a fall, or a stroke. The IMD may initially monitor the patient condition operating as active IMD and may switch to operating as a passive IMD. By switching from operating as an active IMD to operating as a passive IMD, the techniques and devices of this disclosure facilitate more frequent monitoring immediately following a patient episode while still ensuring device longevity and usefulness after the battery life ends. For example, the IMD may be configured to sense patient data to monitor for infection after a surgical procedure. In such examples, infection may be relatively more likely during the first weeks or months after the surgical procedure. Thus, it may be advantageous for the IMD to sense patient data relatively frequently, e.g., continuously, hourly, or daily, in the weeks or months immediately following the surgical procedure. After the first weeks or months, infection risk may be lower for the patient. When the battery life of the IMD ends, the IMD may be configured for passive sensing and/or communication. A user may place the external power source near the implantation, e.g., injection, site of the IMD to provide power to the IMD. The IMD may sense patient data and communicate the patient data. For example, the IMD may communicate the patient data to the external power source, e.g., user smartphone, user tablet, or user remote.

As another example, the IMD may be configured to sense patient data indicative of a risk of a health event, e.g., one or more of a cardiac event risk, a fall risk, or a stroke risk. In some examples, patients who have experienced a serious health event, e.g., one of a cardiac event or a stroke, may be at an increased risk of experiencing another one of the cardiac event or the stroke in the weeks or months following the cardiac event or stroke. Additionally, patients who have experienced a serious health event such as a stroke may be at an increased risk of falling, particularly in the weeks or months following the stroke. By switching from operating as an active IMD to operating as a passive IMD, the techniques of this disclosure may facilitate frequent monitoring of the patient condition in the relatively high risk period of time following a serious health event while still remaining useful for intermittent monitoring after the end of the battery life of the IMD.

As another example, the IMD may be configured to monitor a variety of biomarkers, e.g., chemical biomarkers, after a procedure, e.g., a surgical procedure. The first several days or weeks after the procedure may comprise a first period of time associated with a relatively high risk of infection or other complication, e.g., complications associated with a patient’s condition. During the first period of time, the IMD may perform frequent sensing and communication, e.g., every few minutes, every hour, or every few hours due to the relatively high risk. In some examples, as the first period of time goes on, the frequency of communication may change. For example, during the first period of time, the IMD may switch from communicating on an hourly basis to communicating on a daily basis. During the first period of time, the IMD may communicate via a first, active communication mode. A second period of time after the first period of time may be associated with relatively low risk of infection. The IMD may monitor biomarkers and communicate relatively infrequently, e.g., weekly or monthly, using a second, passive communication mode. In this way, the IMD can continue to monitor biomarkers, even after expiration of a battery of the IMD.

In some examples, the devices, systems, and techniques of this disclosure may allow a user to monitor a patient condition using an injectable IMD for a longer period of time than other devices, systems, and techniques without needing to remove and/or replace the injectable IMD. By increasing the period of time during which the injectable IMD is useful, the techniques of this disclosure may improve a user, e.g., a patient and/or clinician, experience and may improve patient outcomes. For example, the patient and clinician may spend less time in-clinic for follow-up visits. Additionally, monitoring the patient condition for a relatively longer period of time may reduce anxiety for the patient associated with the patient condition. Both the clinician and the patient may be able to spend less time in follow-up clinic visits removing and/or replacing the injectable IMD, thereby reducing clinician burden and improving quality of life for the patient.

According to the foregoing and the following description, the techniques disclosed herein offer substantial technological benefits over the references of record. In the context of IMDs where an expendable power source cannot be easily replaced (as it may require explant and reimplant to the patient), the embodiments and power management strategies disclosed herein are capable of significantly extending the useful life of the device. For example, by enabling the device to switch from an active to a passive mode, a depleted battery need not end the useful life of the device; instead, the device an continue in operation, in some cases indefinitely, even if in some embodiments it is at a reduced-but-still-useful functionality. This may also have the effect of requiring that a patient submit to fewer surgical procedures over the course of their life, as the IMD device may need to be replaced less frequently or not at all. Further, while these benefits including improved lifetime power management are particularly impactful in the context of implantable devices, it will be understood that these are also benefits to other types of devices such as, for example, wearable health monitors taking the form of adhesive patches or wristwatches. Accordingly, while various embodiments are described herein in the context of IMDs, various modifications will be apparent to implement these teachings for managing power and operation of wearable devices or virtually any device that relies on an expendable primary power source to drive its operation. Various additional technological benefits will be apparent in view of the present disclosure.

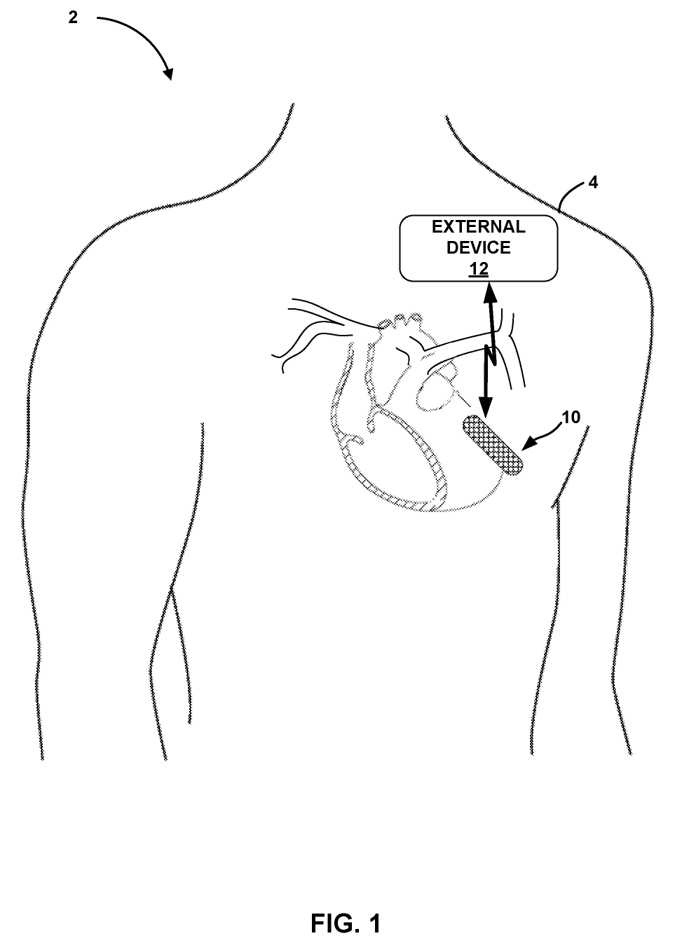

FIG. 1 illustrates the environment of an example medical device system 2 in conjunction with a patient 4, in accordance with one or more techniques of this disclosure. The example techniques may be used with one or more patient sensing devices, e.g., including an IMD 10, which may be in wireless communication with one or more computing devices, e.g., external device 12. Although not illustrated in FIG. 1, IMD 10 may include electrodes and/or other sensors to sense patient data, e.g., one or more physiological signals, of patient 4. The one or more physiological signals may include one or more of a cardiac electrogram (EGM) signal, an impedance signal, an accelerometer signal, e.g., an accelerometer signal indicative of patient posture and/or patient activity level, a temperature signal, an optical signal, or any other physiological signal of patient 4 indicative of a patient condition. In some examples, IMD 10 may be configured to monitor one or more biomarkers, e.g., blood pressure, glucose, hemoglobin A1c, creatinine kinase, cholesterol, and/or cytokines. In some examples, the one or more biomarkers comprise one or more chemical biomarkers. Various approaches for monitoring chemical biomarkers may be utilized such as functional coatings on the device 10 and/or optical monitoring (e.g., of changes in a functional coating or of analytes themselves). IMD 10 may sense data indicative of blood oxygenation, data indicative of impedance, data indicative of temperature, data indicative of posture, data indicative of activity level, and/or chemical sensor data.

IMD 10 may be implanted outside of a thoracic cavity of patient 4 (e.g., subcutaneously in the pectoral location illustrated in FIG. 1). IMD 10 may be positioned near the sternum near or just below the level of the heart of patient 4, e.g., at least partially within the cardiac silhouette, and be configured to sense an ECG and/or other physiological signals from that position. In some examples, IMD 10 takes the form of the Reveal LINQ™ or LINQ II™ ICM. Although described primarily in the context of examples in which IMD 10 takes the form of an ICM implanted near the sternum of patient 4, the techniques of this disclosure may be implemented in systems including any one or more IMDs implanted anywhere in the body of patient 4. For example, IMD 10 may be an injectable IMD configured to be injected at various subcutaneous locations of the body of patient 4.

External device 12 may be a computing device with a display viewable by the user and an interface for providing input to external device 12 (i.e., a user input mechanism). External device 12 is configured for both active and passive wireless communication with IMD 10. External device 12 retrieves sensed physiological data from IMD 10 that was collected and, in some examples, stored, by IMD 10. In some examples, external device 12 takes the form of a personal computing device of patient 4 or a clinician. For example, external device 12 may take the form of a smartphone of patient 4. In some examples, external device 12 may be any computing device configured for wireless communication with IMD 10, such as a desktop, laptop, or tablet computer. External device 12 may communicate with IMD 10 via far-field communication technologies, e.g., radiofrequency telemetry according to the Bluetooth® or Bluetooth® Low Energy (BLE) protocols, or other communication technologies operable at ranges greater than near-field communication technologies during a first period of time in which IMD 10 operates as an active IMD. External device 12 may communicate with IMD 10 via radio frequency identification (RFID) communication, such as near-field communication technologies e.g., inductive coupling, NFC or other communication technologies operable at ranges less than 10–20 cm, during a second period of time in which IMD 10 operates an a passive IMD. In some embodiments, external device 12 may be used to transmit instructions to IMD 10 (e.g., identifying one or more operations for the IMD 10 to perform under passive power such as gathering physiological data for sensors, applying analysis to such physiological data, and/or reporting data to the external device 12 or another device). Such instruction transmission may be particularly useful when the external device 12 is configured for use by the clinician. The user (e.g., theclinician) may also configure and store operational parameters for IMD 10 with the aid of external device 12. In some examples, external device 12 assists the user in the configuration of IMD 10 by providing a system for identifying potentially beneficial operational parameter values. For example, based on the patient condition IMD 10 is configured to monitor and/or additional patient information, external device 12 may identify a periodic schedule for sensing patient data (e.g., a reduced activation schedule chosen to utilize but not exhaust remaining primary power before the IMD 10 is removed, replaced, or otherwise reaches the end of its intended life for the patient).

External device 12 may be used to retrieve data from IMD 10. The retrieved data may include patient data sensed by IMD 10 based on one or more physiological signals sensed by IMD 10. For example, during the first period of time, IMD 10 may communicate sensed patient data to external device 12 using a first type of communication, e.g., BLE communication. IMD 10 may communicate sensed patient data to external device 12 continuously, e.g., according to a periodic schedule, such as an hourly schedule or a daily schedule. The patient data transmission may comprise patient data corresponding to a specified duration of time, e.g., 10 seconds, and/or comprise a specified number of patient data samples.

During the second period of time, external device 12 may provide power to IMD 10 via proximate energy transfer, e.g., NFC. IMD 10 may sense patient data in response to receiving power from external device 12 and/or may communicate sensed patient data to external device 12 in response to receiving power from external device 12. In some examples, external device 12 determines whether the patient data is indicative of a patient condition and/or a change in a patient condition.

In some examples, external device 12 e.g., a smartphone of patient 4, may transmit the data to a computing system via a network. Additionally, or alternatively, IMD 10 may transmit patient data, to the computing system via an access point (not illustrated in FIG. 1). In some examples, IMD 10 may only be configured to transmit patient data to the computing system when IMD 10 is operating as an active device during the first period of time. The computing system may process the patient data and determine whether the patient data is indicative of a patient condition and/or a change in the patient condition.

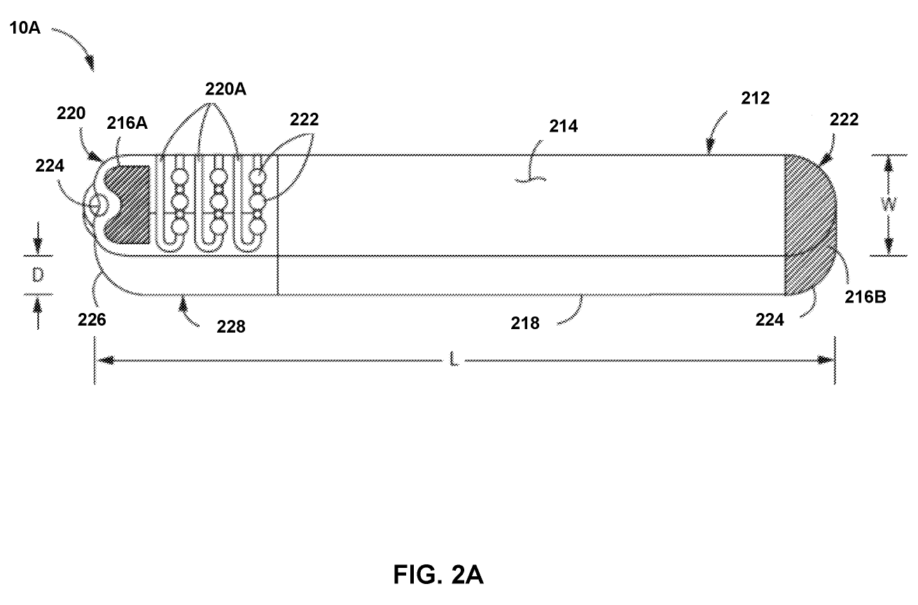

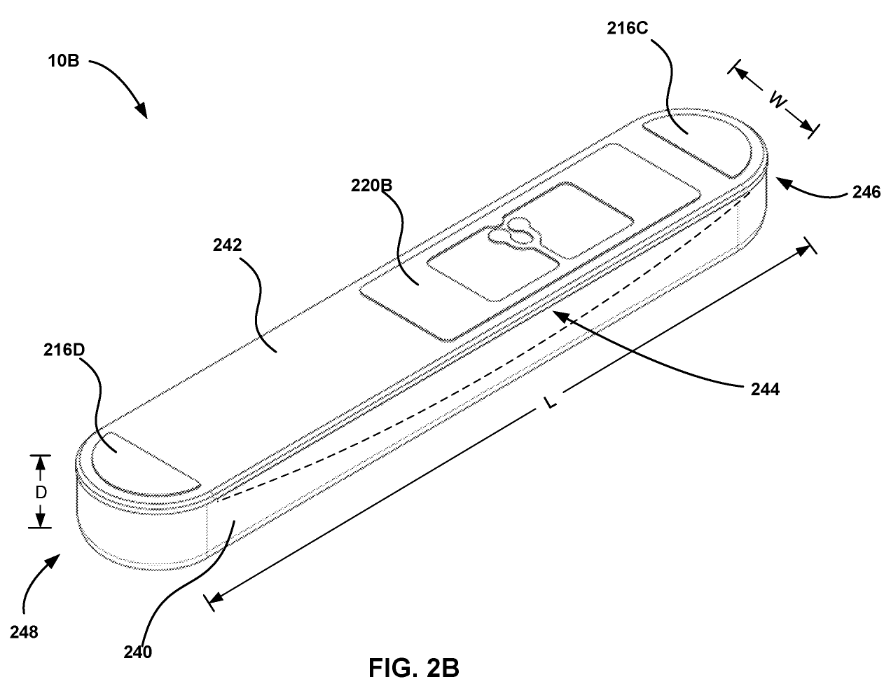

FIG. 2A is a perspective drawing illustrating an IMD 10A, which may be an example configuration of IMD 10 of FIG. 1 as an ICM. In the example shown in FIG. 2A, IMD 10A may be embodied as a monitoring device having housing 212, proximal electrode 216A and distal electrode 216B. Housing 212 may further comprise first major surface 214, second major surface 218, proximal end 220, and distal end 222. Housing 212 encloses electronic circuitry located inside the IMD 10A and protects the circuitry contained therein from body fluids. Housing 212 may be hermetically sealed and configured for subcutaneous implantation. Electrical feedthroughs provide electrical connection of electrodes 216A and 216B.

In the example shown in FIG. 2A, IMD 10A is defined by a length L, a width W and thickness or depth D and is in the form of an elongated rectangular prism wherein the length L is much larger than the width W, which in turn is larger than the depth D. In one example, the geometry of the IMD 10A – in particular a width W greater than the depth D – is selected to allow IMD 10A to be inserted under the skin of the patient using a minimally invasive procedure and to remain in the desired orientation during insertion. For example, the device shown in FIG. 2A includes radial asymmetries (notably, the rectangular shape) along the longitudinal axis that maintains the device in the proper orientation following insertion. For example, the spacing between proximal electrode 216A and distal electrode 216B may range from 5 millimeters (mm) to 55 mm, 20 mm to 55 mm, 25 mm to 55 mm, and from 40 mm to 55 mm and may be any range or individual spacing from 5 mm to 60 mm. In addition, IMD 10A may have a length L that ranges from 20 mm to about 70 mm. In other examples, the length L may range from 5 mm to 60 mm, 40 mm to 60 mm, 45 mm to 60 mm and may be any length or range of lengths between about 20 mm and about 70 mm. In addition, the width W of major surface 214 may range from 2 mm to 15, mm, from 2 mm to 10 mm, or from 5 mm to 15 mm, and may be any single or range of widths between 2 mm and 15 mm. The thickness of depth D of IMD 10A may range from 2 mm to 15 mm, from 2 mm to 9 mm, from 2 mm to 5 mm, from 5 mm to 15 mm, and may be any single or range of depths between 2 mm and 15 mm. In addition, IMD 10A according to an example of the present disclosure is has a geometry and size designed for ease of implant and patient comfort. Examples of IMD 10A described in this disclosure may have a volume of three cubic centimeters (cm) or less, 1.5 cubic cm or less or any volume between three and 1.5 cubic centimeters.

IMD 10A need not take the form of a rectangular prism. As an example, IMD 10A may defined by a diameter D, a length L and is in the form of a cylinder. In some examples, the diameter D may range up to 2 mm or 2.2 mm. The length L may range up to 15 mm.

In the example shown in FIG. 2A, once inserted, e.g., injected, within the patient, the first major surface 214 faces outward, toward the skin of the patient while the second major surface 218 is located opposite the first major surface 214. In addition, in the example shown in FIG. 2A, proximal end 220 and distal end 222 are rounded to reduce discomfort and irritation to surrounding tissue once inserted under the skin of the patient. IMD 10A, including instrument and method for inserting IMD 10A is described, for example, in U.S. Patent No. 11,311,312, incorporated herein by reference in its entirety.

Proximal electrode 216A is at or proximate to proximal end 220, and distal electrode 16B is at or proximate to distal end 222. Proximal electrode 216A and distal electrode 216B are used to sense patient data, e.g., one or more physiological signals, thoracically outside the ribcage, which may be implanted sub-muscularly or subcutaneously. Physiological signals may be stored in a memory of IMD 10A, and data may be transmitted via integrated antenna(e) 220A to another device, which may be another implantable device or an external device, such as external device 212. In some examples, during the first period of time, integrated antenna(e) 220A may be configured for BLE communication. During the second period of time, integrated antenna 220A may be configured for RFID communication and/or NFC communication. In some examples, integrated antenna 220A is configured to receive power from an external power source, e.g., external device 12. In some examples, IMD 10A includes more than one integrated antenna 220A. A first integrated antenna 220A may be configured for a first type of communication, e.g., BLE communication, and a second integrated antenna 220A may be configured for a second type of communication, e.g., NFC. In some examples, the second type of communication may operate at 915 megahertz (MHz).

In some examples, electrodes 216A and 216B may be used for sensing any physiological signal of interest, which may be, for example, a cardiac EGM signal, an electroencephalogram (EEG) signal, an electromyogram (EMG) signal, etc. In some examples, IMD 10A may be configured to sense a temperature signal, an optical signal, a nerve signal, an accelerometer signal, e.g., an accelerometer signal indicative of patient activity level and/or patient posture, or an impedance, from any implanted location. In some examples, IMD 10A may be configured to monitor one or more biomarkers. In some examples, the one or more biomarkers comprise one or more chemical biomarkers. IMD 10A may sense data indicative of blood oxygenation, data indicative of impedance, data indicative of temperature, data indicative of posture, data indicative of activity level, and/or chemical sensor data.

In the example shown in FIG. 2A, proximal electrode 216A is at or in close proximity to the proximal end 220 and distal electrode 216B is at or in close proximity to distal end 222. In this example, distal electrode 216B is not limited to a flattened, outward facing surface, but may extend from first major surface 214 around rounded edges 224 and/or end surface 226 and onto the second major surface 218 so that the electrode 216B has a three-dimensional curved configuration. In some examples, electrode 216B is an uninsulated portion of a metallic, e.g., titanium, part of housing 212.

In the example shown in FIG. 2A, proximal electrode 216A is located on first major surface 214 and is substantially flat, and outward facing. However, in other examples proximal electrode 216A may utilize the three-dimensional curved configuration of distal electrode 216B, providing a three-dimensional proximal electrode (not shown in this example). Similarly, in other examples distal electrode 216B may utilize a substantially flat, outward facing electrode located on first major surface 214 similar to that shown with respect to proximal electrode 216A.

The various electrode configurations allow for configurations in which proximal electrode 216A and distal electrode 216B are located on both first major surface 214 and second major surface 218. In other configurations, such as that shown in FIG. 2A, only one of proximal electrode 216A and distal electrode 216B is located on both major surfaces 214 and 218, and in still other configurations both proximal electrode 216A and distal electrode 216B are located on one of the first major surface 214 or the second major surface 218 (e.g., proximal electrode 216A is located on first major surface 214 while distal electrode 216B is located on second major surface 218). In another example, IMD 10A may include electrodes on both major surface 214 and 218 at or near the proximal and distal ends of the device, such that a total of four electrodes are included on IMD 10A. Electrodes 216A and 216B may be formed of a plurality of different types of biocompatible conductive material, e.g., stainless steel, titanium, platinum, iridium, or alloys thereof, and may utilize one or more coatings such as titanium nitride or fractal titanium nitride.

In the example shown in FIG. 2A, proximal end 220 includes a header assembly 228 that includes one or more of proximal electrode 216A, integrated antenna(e) 220A, anti-migration projections 222, and/or suture hole 224. Integrated antenna(e) 220A is located on the same major surface (i.e., first major surface 214) as proximal electrode 216A and is also included as part of header assembly 228. Integrated antenna(e) 220A allows IMD 10A to transmit and/or receive data during both the first period of time and the second period of time. In other examples, integrated antenna(e) 220A may be formed on the opposite major surface as proximal electrode 216A or may be incorporated within the housing 212 of IMD 10A. In the example shown in FIG. 2A, anti-migration projections 222 are located adjacent to integrated antenna(e) 220A and protrude away from first major surface 214 to prevent longitudinal movement of the device. In the example shown in FIG. 2A, anti-migration projections 222 include a plurality of small bumps or protrusions (e.g., nine small bumps) extending away from first major surface 214. As discussed above, in other examples anti-migration projections 222 may be located on the opposite major surface as proximal electrode 216A and/or integrated antenna(e) 220A. In addition, in the example shown in FIG. 2A, header assembly 228 includes suture hole 224, which provides another means of securing IMD 10A to the patient to prevent movement following insertion. In the example shown, suture hole 224 is located adjacent to proximal electrode 216A. In one example, header assembly 228 is a molded header assembly made from a polymeric or plastic material, which may be integrated or separable from the main portion of IMD 10A.

FIG. 2B is a perspective drawing illustrating another IMD 10B, which may be another example configuration of IMD 10 from FIG. 1 as an ICM. IMD 10B of FIG. 2B may be configured substantially similarly to IMD 10A of FIG. 2A, with differences between them discussed herein.

IMD 10B may include a leadless, subcutaneously-implantable monitoring device, e.g., an ICM. IMD 10B includes housing having a base 240 and an insulative cover 242. Proximal electrode 216C and distal electrode 216D may be formed or placed on an outer surface of cover 242. Various circuitries and components of IMD 10B may be formed or placed on an inner surface of cover 242, or within base 240. In some examples, a battery or other power source of IMD 10B may be included within base 240. In the illustrated example, antenna(e) 220B are formed or placed on the outer surface of cover 242 but may be formed or placed on the inner surface in some examples. In some examples, insulative cover 242 may be positioned over an open base 240 such that base 240 and cover 242 enclose the circuitries and other components and protect them from fluids such as body fluids. The housing including base 270 and insulative cover 272 may be hermetically sealed and configured for subcutaneous implantation.

Circuitries and components may be formed on the inner side of insulative cover 242, such as by using flip-chip technology. Insulative cover 242 may be flipped onto a base 240. When flipped and placed onto base 240, the components of IMD 10B formed on the inner side of insulative cover 242 may be positioned in a gap 244 defined by base 240. Electrodes 216C and 216D and antenna 220B may be electrically connected to circuitry formed on the inner side of insulative cover 242 through one or more vias (not shown) formed through insulative cover 242. Insulative cover 242 may be formed of sapphire (i.e., corundum), glass, parylene, and/or any other suitable insulating material. Base 240 may be formed from titanium or any other suitable material (e.g., a biocompatible material). Electrodes 216C and 216D may be formed from any of stainless steel, titanium, platinum, iridium, or alloys thereof. In addition, electrodes 216C and 216D may be coated with a material such as titanium nitride or fractal titanium nitride, although other suitable materials and coatings for such electrodes may be used.

In the example shown in FIG. 2B, the housing of IMD 10B defines a length L, a width W and thickness or depth D and is in the form of an elongated rectangular prism wherein the length L is much larger than the width W, which in turn is larger than the depth D, similar to IMD 10A of FIG. 2A. For example, the spacing between proximal electrode 216C and distal electrode 216D may range from 5 mm to 50 mm, from 20 mm to 50 mm, from 25 mm to 45 mm, and may be any single spacing or range of spacings from 5 mm to 50 mm, such as approximately 40 mm. In addition, IMD 10B may have a length L that ranges from 5 mm to about 70 mm. In other examples, the length L may range from 20 mm to 70 mm, 40 mm to 60 mm, 45 mm to 55 mm, and may be any single length or range of lengths from 5 mm to 50 mm, such as approximately 45 mm. In addition, the width W may range from 2 mm to 15 mm, 5 mm to 15 mm, 5 mm to 10 mm, and may be any single width or range of widths from 2 mm to 15 mm, such as approximately 8 mm. The thickness or depth D of IMD 10B may range from 2 mm to 15 mm, from 5 mm to 15 mm, or from 2 mm to 5 mm, and may be any single depth or range of depths between 2 mm and 15 mm, such as approximately 4 mm. IMD 10B may have a volume of three cubic centimeters (cm) or less, or 1.5 cubic cm or less, such as approximately 1.4 cubic cm.

In the example shown in FIG. 2B, once inserted subcutaneously within the patient, outer surface of cover 242 faces outward, toward the skin of the patient. In addition, as shown in FIG. 2B, proximal end 246 and distal end 248 are rounded to reduce discomfort and irritation to surrounding tissue once inserted under the skin of the patient. In addition, edges of IMD 10B may be rounded.

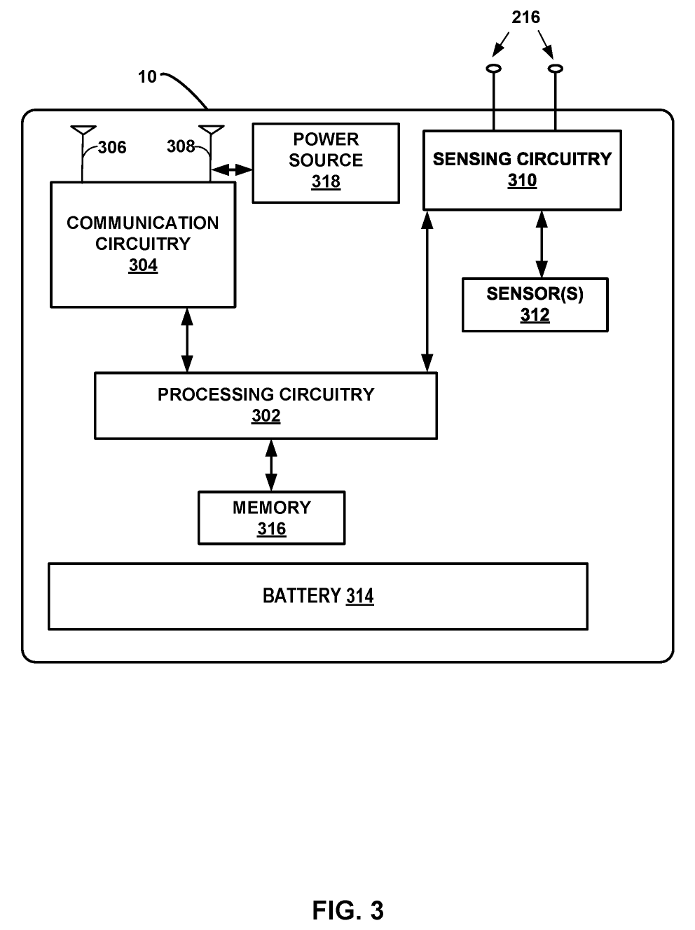

FIG. 3 is a block diagram illustrating an example configuration of an IMD 10 in accordance with one or more techniques described herein. IMD 10 may correspond to either of IMDs 10A and 10B, or another configuration of an IMD. In the illustrated example, IMD 10 includes electrodes 216 (which may correspond to any of electrodes 216A–216D), processing circuitry 302, sensing circuitry 310, sensors 312, communication circuitry 304, battery 314, power source 318, and memory 316. Although the illustrated example includes two electrodes 216, IMDs including or coupled to more than two electrodes or less than two electrodes, e.g., no electrodes, may implement the techniques of this disclosure in some examples. Battery 314 provides operational power for processing circuitry 302, sensing circuitry 310, sensors 312, communication circuitry 304, and memory 316.

Battery 314 may be a primary cell battery. Battery 314 may provide operational power for processing circuitry 302, sensing circuitry 310, sensors 312, communication circuitry 304, and memory 316 for a first period of time. The first period of time may correspond to the lifetime of battery 314. During a second period of time, one or more of processing circuitry 302, sensing circuitry 310, sensors 312, communication circuitry 304, and/or memory 316 may receive operational power from an external power source, e.g., external device 12.

While various embodiments are described herein as relying on a battery as a primary power source, it will be apparent that various additional or alternative components may be used as a primary power source. For example, in addition to or instead of battery 314, one or more capacitors (not shown) may be included in the device 10 to provide electric power to the other components. In some embodiments, the device 10 may include means to recharge the primary power source(s) such as through energy harvesting (e.g., capturing heat, motion, or chemical energy from the patient and converting to electrical energy) or from induced power (e.g., from the external device described herein, another external device, or another device implanted in the patient). As described, the primary power source is entirely internal to the device. In some embodiments, the primary power source may be at least partially external to the device 10. For example, another implant in the patient may be configured to continuously power the device 10 in its active state, e.g., via induced power. In such embodiments, the techniques described herein for switching the operating mode of the device 10 (e.g., switching the communication mode) may be employed once such an external primary power source is sufficiently depleted (e.g., falls below a threshold). In such embodiments, the circuitry or other components of the device adapted to receive power from an external primary power source may constitute an internal power source of the device 10 but is not fully internal as it relies on power external to the device to operate.

Processing circuitry 302 may include fixed function circuitry and/or programmable processing circuitry. Processing circuitry 302 may include any one or more of a microprocessor, a controller, a DSP, an ASIC, a FPGA, a GPU, a TPU, or equivalent discrete or analog logic circuitry. In some examples, processing circuitry 302 may include multiple components, such as any combination of one or more microprocessors, one or more controllers, one or more DSPs, one or more ASICs, or one or more FPGAs, one or more GPUs, one or more TPUs, as well as other discrete or integrated logic circuitry. The functions attributed to processing circuitry 302 herein may be embodied as software, firmware, hardware or any combination thereof.

Sensing circuitry 310 may be coupled to electrodes 216 to sense patient data of patient 4, for example by selecting electrodes 216 and polarity, used to sense a cardiac EGM as controlled by processing circuitry 302. In examples in which IMD 10 comprises an ICM, sensing circuitry 310 may sense the cardiac EGM from electrodes 216 in order to facilitate monitoring the electrical activity of the heart. In some examples, sensing circuitry 310 may include one or more filters and amplifiers for filtering and amplifying signals received from electrodes 216 and/or sensors 312. In some examples, sensing circuitry 310 and processing circuitry 302 may store patient data in memory 316. Sensing circuitry 310 may monitor signals from sensors 312, which may include one or more accelerometers, other vibration or motion sensors, optical sensors, or blood pressure (BP) sensors, as examples. Sensing circuitry 310 may capture sensor signals from any one of sensors 312, e.g., to produce patient data, in order to facilitate monitoring of the patient condition.

Communication circuitry 304 may include any suitable hardware, firmware, software, or any combination thereof for communicating with another device, such as external device 12, another networked computing device, or another IMD or sensor. Under the control of processing circuitry 302, communication circuitry 304 may receive downlink telemetry from, as well as send uplink telemetry to external device 12. In addition, processing circuitry 302 may communicate with a networked computing device via an external device (e.g., external device 12) and a computer network, such as the Medtronic CareLink™ Network. Communication circuitry 304 may be configured to transmit and/or receive signals via inductive coupling, electromagnetic coupling, NFC, Radio Frequency (RF) communication, Bluetooth, Wi-Fi, or other proprietary or non-proprietary wireless communication schemes.

In some examples, communication circuitry 304 includes one or more of antenna 306 or antenna 308. Antenna 306 may be configured for a first type of communication, e.g., BLE communication. Antenna 308 may be configured for a second type of communication, e.g., NFC communication. Antenna 308 may be configured to operate at a frequency of 915 MHz. In some examples, IMD 10 may be configured to receive power via antenna 308 from an external power source, e.g., external device 12. Power source 318 may retrieve power received via antenna 308. Power source 318 may provide operational power to one or more of communication circuitry 304, sensing circuitry 310, processing circuitry 302, or memory 316. Power source 318 may include one or more rectifiers, capacitors, and/or regulators. In some examples, antenna 308 receives an alternating current voltage. Power source 318 may rectify the alternating current to a direct current voltage. Power source 318 may include a regulator configured to provide stable voltage to the one or more of communication circuitry 304, sensing circuitry 310, processing circuitry 302, or memory 316. Various additional techniques for designing circuitry for receiving induced electric power and subsequently making the power available to other electronic component (e.g., according to passive near field communication schemes) will be apparent.

Communication circuitry 304 may communicate using antenna 306 to perform the first type of communication during a first period of time. Battery 314, e.g., a primary cell battery, may provide operational power to antenna 306. The first period of time may correspond to the lifetime of the battery. During a second period of time, one or more of processing circuitry 302, sensing circuitry 310, sensors 312, communication circuitry 304, and/or memory 316 may receive operational power from external device 12 via antenna 308, which may be configured for RFID, e.g., NFC. In some examples, IMD 10 includes only one antenna configured to facilitate both the first type of communication and the second type of communication.

Memory 316 may be configured to store information within IMD 10 during operation. Memory 316, in some examples, is described as a computer-readable storage medium. In some examples, memory 316 is a temporary memory, meaning that a primary purpose of memory 316 is not long-term storage. Memory 316, in some examples, is described as a volatile memory, meaning that memory 316 does not maintain stored contents when the computer is turned off. Examples of volatile memories include RAM, dynamic RAM (DRAM), static RAM (SRAM), and other forms of volatile memories known in the art. In some examples, memory 316 is used to store program instructions for execution by processing circuitry 302. Memory 316, in one example, is used by software or applications running on IMD 10 to temporarily store information during program execution.

Memory 316, in some examples, also includes one or more non-transitory computer-readable storage media. Memory 316 may be configured to store larger amounts of information than volatile memory. Memory 316 may further be configured for long-term storage of information. In some examples, memory 316 includes non-volatile storage elements. Examples of such non-volatile storage elements include magnetic hard discs, optical discs, flash memories, or forms of electrically programmable memories (EPROM) or EEPROM memories.

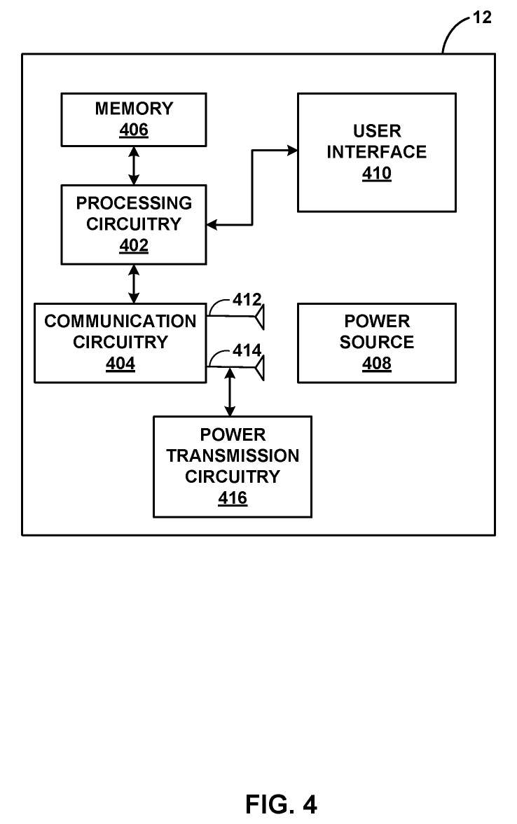

FIG. 4 is a block diagram illustrating an example configuration of external device 12 of FIG. 1 that operates in accordance with one or more techniques of this disclosure. While external device 12 may generally be described as a hand-held computing device, external device 12 may be a notebook computer, a smart phone, a workstation, a key fob, or a wearable device, for example. As illustrated in FIG. 4, external device 12 may include a processing circuitry 402, memory 406, user interface 410, communication circuitry 404, power transmission circuitry 416, and power source 408. Memory 406 may store program instructions that, when executed by processing circuitry 402, cause processing circuitry 402 and external device 12 to provide the functionality ascribed to external device 12 throughout this disclosure.

In general, external device 12 comprises any suitable arrangement of hardware, alone or in combination with software and/or firmware, to perform the techniques attributed to external device 12, and processing circuitry 402, user interface 410, and communication circuitry 404 of external device 12. In various examples, external device 12 may include one or more processors, such as one or more microprocessors, DSPs, ASICs, FPGAs, or any other equivalent integrated or discrete logic circuitry, as well as any combinations of such components. External device 12 also, in various examples, may include a memory 406, such as RAM, ROM, PROM, EPROM, EEPROM, flash memory, a hard disk, a CD-ROM, comprising executable instructions for causing the one or more processors to perform the actions attributed to them. Moreover, although processing circuitry 402 and communication circuitry 404 are described as separate circuitry, in some examples, processing circuitry 402, and communication circuitry 404 are functionally integrated. In some examples, processing circuitry 402 and communication circuitry 404 and communication circuitry 404 correspond to individual hardware units, such as microprocessors, ASICs, DSPs, FPGAs, or other hardware units. In other examples, any of processing circuitry 402, communication circuitry 404, communication circuitry 304 may correspond to multiple individual hardware units, such as microprocessors, ASICs, DSPs, FPGAs, or other hardware units.

Memory 406 may store program instructions that, when executed by processing circuitry 402, cause processing circuitry 402 and external device 12 to provide the functionality ascribed to external device 12 throughout this disclosure. Memory 406 may be configured to store information received from IMD 10. In some examples, memory 406 is used to store program instructions for execution by processing circuitry 402. Memory 406, in one example, is used by software or applications running on external device 12 to store information during program execution.

Memory 406, in some examples, also includes one or more non-transitory computer-readable storage media. Memory 406 may be configured to store larger amounts of information than volatile memory. Memory 406 may further be configured for long-term storage of information. In some examples, memory 406 includes non-volatile storage elements. Examples of such non-volatile storage elements include magnetic hard discs, optical discs, floppy discs, flash memories, or forms of electrically programmable memories (EPROM) or EEPROM memories.

In certain examples, external device 12 includes a user interface 410 that outputs patient condition information to the user, e.g., patient 4 and/or the clinician. During the first period of time, IMD 10 may communicate patient data indicative of a patient condition to external device 12 continuously and/or according to a periodic schedule, e.g., daily. During the second period of time, IMD 10 may, in response to receiving operating power from external device 12, communicate patient data to external device 12.

User interface 410 may include a button or keypad, lights, a speaker for voice commands, a microphone, a turnable knob, a display, such as a liquid crystal (LCD), light-emitting diode (LED), or cathode ray tube (CRT). In some examples the display may be a touch screen. As discussed in this disclosure, processing circuitry 402 may present and receive information relating to a patient condition of patient 4 via user interface 410.

Processing circuitry 402 may also present information to the patient in the form of alerts related to the patient condition and/or a monitoring schedule of the user, e.g., patient 4, the clinician, or a caregiver, via user interface 410. Although not shown, external device 12 may additionally or alternatively include a data or network interface to another computing device, to facilitate communication with the other device, and presentation of information relating to the patient condition via the other device.

Communication circuitry 404 supports wireless communication between IMD 10 and external device 12. Communication circuitry 404 may also be configured to communicate with another computing device via wireless communication techniques, or direct communication through a wired connection. In some examples, communication circuitry 404 may be substantially similar to communication circuitry 304 of IMD 10 described above, providing wireless communication via an RF and/or inductive coupling. In some examples, communication circuitry 404 may include antenna 412 and antenna 414, each of which may take on a variety of forms, such as an internal or external antenna. Antenna 412 may be configured for a first type of communication, e.g., BLE communication. Antenna 414 may be configured for a second type of communication, e.g., NFC. Communication circuitry 404 of external device 12 may be configured to communicate with IMD 10 using the first type of communication using antenna 412 during the first period of time. External device 12 may switch to communicating with IMD 10 using the second type of communication using antenna 414 during the second period of time. In some examples, external device 12 includes only one antenna configured to facilitate both the first type of communication and the second type of communication. In some examples, e.g., during the second period of time, a user may initiate sensing of patient data by placing external device 12 proximate to an implantation site of IMD 10. In such examples, external device 12 may include power transmission circuitry 416. Power transmission circuitry 416 may provide operational power to IMD 10 via antenna 414. As an example, during the second period of time, power transmission circuitry 416 may, via inductive coupling, provide power to IMD 10 via antenna 414. Various techniques for designing circuitry for delivering inductive power to another device (e.g., according to various passive near field communication schemes) will be apparent. The user my place external device 12 proximate to the implantation site of IMD 10 to initiate operation of IMD 10, e.g., sensing by sensing circuitry 310 of IMD 10, and to communicate with IMD 10.

In examples in which the user is patient 4 or a caregiver of patient 4, the user may initiate sensing at times when patient 4 is feeling anxious. In some examples, external device 12 may notify the user, e.g., patient 4, that it is time for a scheduled check-in. In examples in which the user is a clinician, the clinician may initiate sensing during follow-up clinic visits.

External device 12 may additionally be configured to communicate with one or more other computing devices. Examples of local wireless communication techniques that may be employed to facilitate communication between external device 12 and another computing device include RF communication according to the 802.11 or Bluetooth specification sets, e.g., BLE specification sets, infrared communication, e.g., according to the IrDA standard, or other standard or proprietary telemetry protocols. In this manner, other external devices may be capable of communicating with external device 12 without needing to establish a secure wireless connection.

Power source 408 delivers operating power to the components of external device 12. Power source 408 may include a battery and a power generation circuit to produce the operating power. In some examples, the battery may be rechargeable to allow extended operation.

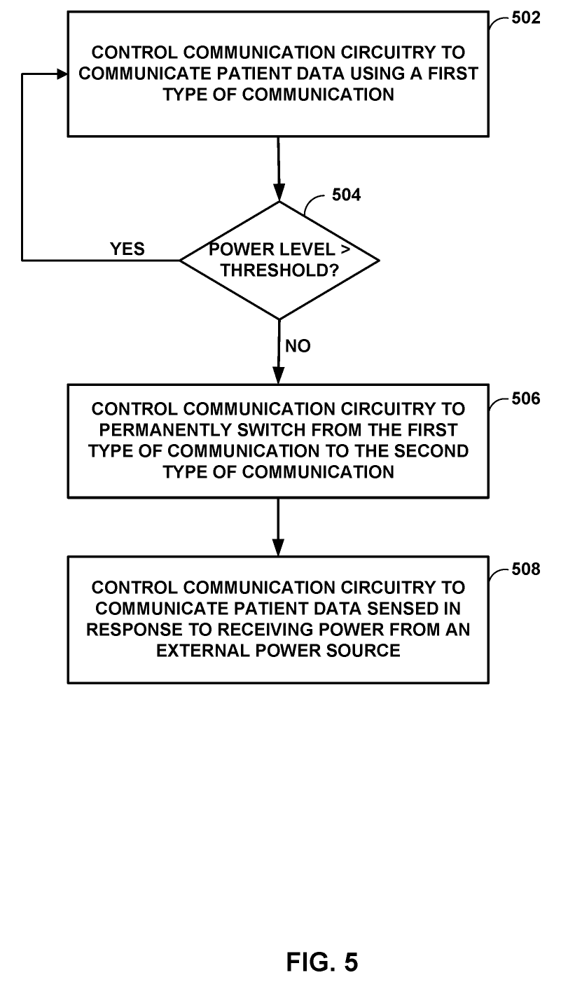

FIG. 5 is a flow diagram illustrating an example operation for permanently switching from communicating using a first type of communication to communicating using a second type of communication, in accordance with one or more techniques of this disclosure. Processing circuitry 302 controls communication circuitry 304 to communicate patient data using a first type of communication (502). The first type of communication may be any type of wireless communication, such as Bluetooth or BLE communication. Battery 314, e.g., a primary cell battery, may provide operational power to communication circuitry 304. If the power level is greater than the threshold, e.g., if the battery life has not ended, (“YES” of 504), processing circuitry 302 continues to control communication circuitry 304 to communicate using the first type of communication (502). In some examples, the threshold value is a threshold amount of usable battery power remaining. The threshold amount may correspond to an amount of usable battery power remaining e.g., when the life of the battery has ended. Processing circuitry 302 controls communication circuitry 304 to communicate using the first type of communication for a first period of time corresponding to the lifetime of the battery. In some examples, when processing circuitry 302 is controlling communication circuitry 304 to communicate using the first type of communication, processing circuitry 302 controls sensing circuitry 310 to sense patient data according to a periodic schedule, e.g., hourly or daily. In some examples, sensing circuitry 310 senses patient data continuously. In some examples, communication circuitry 304 communicates the sensed patient data on a periodic schedule, e.g., hourly or daily.

If the power level is not greater than the threshold (“NO” of 504), processing circuitry 302 controls communication circuitry 304 to permanently switch from communicating using the first type of communication to communicating using a second type of communication, such as for a second period of time following the first period of time (506). The second type of communication may be RFID, e.g., NFC. In some examples, antenna 308 is configured to receive operational power from an external power source, e.g., external device 12. Processing circuitry 302 controls communication circuitry 304 communicates sensed patient data via antenna 308 (508). In some examples, sensing circuitry 310 senses patient data in response to antenna 308 receiving operational power from the external power source.

While various embodiments are described wherein the communication mode is switched as a method of managing power consumption, it will be apparent that alternative or additional changes to the operating mode may be employed to address the depleted primary power source. For example, an overall mode of the device may be switched from an fully active operation mode in step 502 to a fully passive operation mode in step 506. In the fully active operation mode, the primary power source may be used to power the various components of the device (e.g., including the sensors, processing circuitry, and communication circuitry) while in the fully passive operation mode the components of the device may remain unpowered and inoperative until powered by an external device. In this fully passive mode, power from the external device may provide enough power for the device to make a physiological measurement, perform an analysis, and/or report information to the external device. Thus, for example, the device, when in fully passive mode, can still be used to obtain new measurements from the patient by providing the external power. As another example, rather than moving to a fully passive operation mode in step 506, the device may be placed in a partial passive operation mode. According to the partial passive operation mode, power may be provided only to some components and/or one or more components may be configured to not perform certain functionality despite being operative. For example, while in a partial passive mode, an optical sensor may be fully turned off while EGM electrodes may still receive power. As another example, while in a partial passive mode, the processing circuitry may be configured to gather and store EGM data for the patient but not to perform one or more advanced analysis algorithms such as algorithms for identifying various arrhythmias indicated by the EGM data. In some embodiments, while a device is in passive mode (whether full or partial), temporarily providing the external power source may reactivate one or more components or functions of the device. For example, provision of the external power source may temporarily reactivate all components and functions deactivated when switching away from active operation mode. As another example, only some components or functions may be reactivated- for example, the processing circuitry may be activated to perform advanced analysis, to identify arrhythmias while the optical sensor remains deactivated. Various schemes for determining which components and functions to restore when the external power is applied in a passive mode will be apparent.

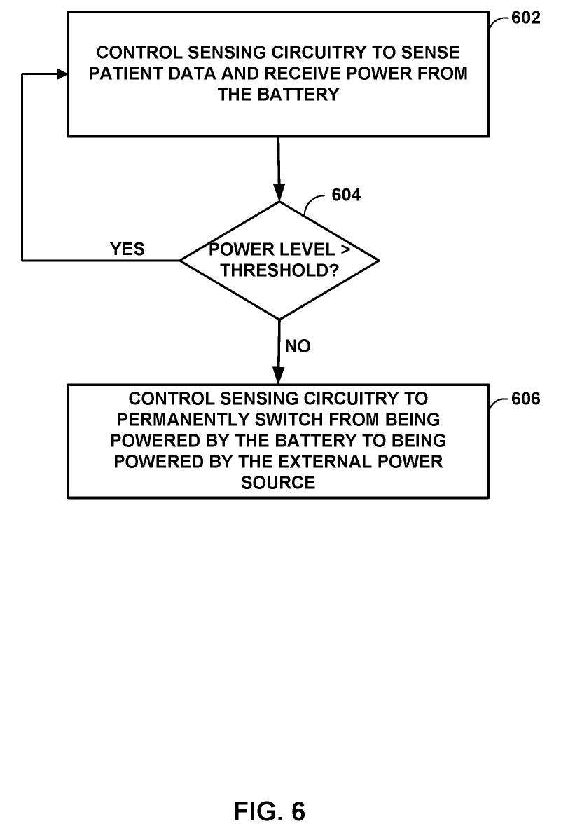

FIG. 6 is a flow diagram illustrating an example operation for permanently switching from sensing according to a periodic schedule via the battery to sensing in response to receiving power from an external power source, in accordance with one or more techniques of this disclosure. Processing circuitry 302 controls sensing circuitry 310 to sense patient data and receive operational power from battery 314, e.g., a primary cell battery (602). Sensing circuitry 310 may sense patient data according to a periodic schedule, e.g., hourly or daily. In some examples, sensing circuitry 310 senses patient data continuously. If a power level of the battery is greater than a threshold power level (“YES” of 604), processing circuitry 302 continues to control sensing circuitry 310 to sense patient data and receive power from the battery (602).

If the power level of the battery is not greater than the threshold (“NO” of 604), processing circuitry 302 permanently switches from controlling sensing circuitry 310 to be powered the battery to being powered by an external power source, e.g., external device 12 (606). In some examples, the threshold value is a threshold amount of usable battery power remaining. The threshold amount of usable battery power remaining may correspond to the usable battery power remaining when the life of the battery has ended. In some examples, sensing circuitry 310 senses patient data in response to receiving power from the external power source. Sensing circuitry 310 may sense a threshold number of samples of patient data and/or sense the patient data for a threshold period of time, e.g., 10 seconds. Communication circuitry 304 may communicate the patient data to external device 12. In some examples, a user may initiate sensing of patient data by placing external device 12 proximate to an implantation site of IMD 10. In examples in which the user is patient 4 or a caregiver of patient 4, the user may initiate sensing at times when patient 4 is feeling anxious. In some examples, external device 12 may notify the user, e.g., patient 4, that it is time for a scheduled check. In examples in which the user is a clinician, the clinician may initiate sensing during follow-up clinic visits.

The following is a non-limiting list of examples that are in accordance with one or more techniques of this disclosure.

Example 1. An implantable medical device (IMD) comprising: a primary power source; sensing circuitry; communication circuitry configured to communicate via a first type of communication and a second type of communication, the first type of communication being facilitated by the primary power source and the second type of communication being facilitated via an external power source; and processing circuitry configured to: control the communication circuitry to communicate patient data sensed via the sensing circuitry using the first type of communication when a power level of the primary power source exceeds a threshold power level; control the communication circuitry to permanently switch from the first type of communication to the second type of communication when the power level of the primary power source falls below the threshold power level; and control the communication circuitry to communicate patient data sensed in response to receiving power from the external power source.

Example 2. The IMD of example 1, wherein the processing circuitry is configured to: control the sensing circuitry to sense patient data and to receive power from the primary power source when the power level of the primary power source exceeds the threshold power level; control the sensing circuitry to permanently switch from being powered by the primary power source to being powered by the external power source and sensing patient data in response to receiving energy from the external power source when the power level of the primary power source falls below the threshold power level.

Example 3. The IMD of example 1 or 2, wherein the communication circuitry is configured to communicate via the first type of communication using a first communication component and communicate via the second type of communication using a second communication component.

Example 4. The IMD of any of examples 1–2, wherein the first type of communication comprises Bluetooth low energy (BLE) communication.

Example 5. The IMD of any of examples 1–4, wherein the second type of communication comprises radio frequency identification (RFID) communication.

Example 6. The IMD of example 5, wherein the RFID communication comprises near field communication (NFC).

Example 7. The IMD of any of examples 1–6, wherein the threshold power level corresponds to expiration of the primary power source.

Example 8. The IMD of any of examples 1–7, wherein the processing circuitry is configured to control the sensing circuitry to sense patient data according to a periodic schedule using the first type of communication when the power level of the primary power source exceeds the threshold power level.

Example 9. The IMD of any of examples 1–8, wherein the external power source is a user device configured to receive the patient data.

Example 10. The IMD of any of examples 1–9, wherein the IMD is injectable.

Example 11. The IMD of any of examples 1–10, wherein the patient data comprises one or more sensed physiological signals.

Example 12. The IMD of example 11, wherein the one or more sensed physiological signals include one or more of: a cardiac electrogram; an impedance; a chemical biomarker; or an accelerometer signal.

Example 13. A method comprising: controlling, by processing circuitry of an implantable medical device (IMD), communication circuitry of the IMD to communicate patient data sensed via sensing circuitry of the IMD using a first type of communication when a power level of a primary power source of the IMD exceeds a threshold power level; controlling, by the processing circuitry, the communication circuitry to permanently switch from the first type of communication to a second type of communication when the power level of the primary power source falls below the threshold power level; and controlling, by the processing circuitry, the communication circuitry to communicate patient data sensed in response to receiving power from the external power source.

Example 14. The method of example 13, further comprising: controlling, by the processing circuitry, the sensing circuitry to sense patient data and to receive power from the primary power source when the power level of the primary power source exceeds the threshold power level; and controlling, by the processing circuitry, the sensing circuitry to permanently switch from being powered by the primary power source to being powered by the external power source and sensing patient data in response to receiving energy from the external power source when the power level of the primary power source falls below the threshold power level.

Example 15. The method of example 13 or 14, wherein the communication circuitry is configured to communicate via first type of communication using a first communication component and communicate via the second type of communication using a second communication component.

Example 16. The method of any of examples 13–15, wherein the first type of communication comprises Bluetooth low energy (BLE) communication.

Example 17. The method of any of examples 13–16, wherein the second type of communication comprises radio frequency identification (RFID) communication.

Example 18. The method of example 17, wherein the RFID communication comprises near field communication (NFC).

Example 19. The method of any of examples 13–18, wherein the threshold power level corresponds to expiration of the primary power source.

Example 20. The method of any of examples 13–19, wherein controlling the sensing circuitry to sense patient data using the first type of communication comprises: controlling, by the processing circuitry, the sensing circuitry to sense patient data according to a periodic schedule using the first type of communication when the power level of the primary power source exceeds the threshold power level.

Example 21. The method of any of examples 13–20, wherein the external power source is a user device configured to receive the patient data.

Example 22. The method of any of examples 13–21, wherein the IMD is injectable.

Example 23. The method of any of examples 13–22, wherein the patient data comprises one or more sensed physiological signals.

Example 24. The method of example 23, wherein the one or more sensed physiological signals include one or more of: a cardiac electrogram; an impedance; a chemical biomarker; or an accelerometer signal.

Example 25. A non-transitory computer-readable medium storing instructions that when executed cause processing circuitry to: control communication circuitry of an implantable medical device (IMD) to communicate patient data sensed via sensing circuitry of the IMD using a first type of communication when a power level of a primary power source of the IMD exceeds a threshold power level; control the communication circuitry to permanently switch from the first type of communication to a second type of communication when the power level of the primary power source falls below the threshold power level; and control the communication circuitry to communicate patient data sensed in response to receiving power from the external power source.

Example 26. A patient monitoring device comprising: a primary power source; external power circuitry configured to receive power from an external power source; sensing circuitry configured to measure at least one physiological parameter of a patient; memory for storing data corresponding to measurements of the at least one physiological parameter; and processing circuitry configured to: monitor a state of the primary power source to determine when the primary power source reaches a depleted state; based on determining that the primary power source is in a depleted state, switch operation of at least one component of the patient monitoring device to a passive operation mode, wherein the at least one component operates at a reduced functionality when an external power source is not in communication with the external power circuitry.

Example 27. An electronic device comprising: a primary power source; external power circuitry configured to receive power from an external power source and processing circuitry configured to: monitor a state of the primary power source to determine when the primary power source reaches a depleted state; based on determining that the primary power source is in a depleted state, switch operation of at least one component of the patient monitoring device to a passive operation mode, wherein the at least one component operates at a reduced functionality when an external power source is not in communication with the external power circuitry.

Example 28. The device of example 26 or 27, further comprising: communication circuitry for communicating with at least one of the external power source and another external device, wherein, in switching operation of the at least one component, the processing circuitry is configured to switch the communication circuitry from using a first type of communication driven by the primary power source to using a second type of communication driven by the external power circuitry.

Example 29. The device of example 26 or 27, wherein, in determining that the primary power source has reached a depleted state, the processing circuitry is configured to compare a power level of the primary power source to a threshold.

Example 30. The device of example 26 or 27, wherein the primary power source is fully internal to the patient monitoring device.

Example 31. The device of example 26 or 27, wherein the at least one component comprises at least one of the sensing circuitry and the processing circuitry.

Example 32. The device of example 26 or 27, wherein the passive operation mode is a fully passive operation mode wherein all functionality of the device is stopped when an external power source is not in communication with the external power circuitry.

Example 33. The device of example 26 or 27, wherein the passive operation mode is a partially passive operation mode wherein a first subset of functionality of the device is stopped and a second subset of functionality of the device continues when an external power source is not in communication with the external power circuitry.

Example 34. The device of example 26 or 27, wherein the passive operation mode is a partially passive operation mode wherein at least some functionality of the device is performed on a reduced activation schedule when an external power source is not in communication with the external power circuitry.

Various examples have been described. These and other examples are within the scope of the following claims.

1. An implantable medical device (IMD) comprising:
a primary power source;
sensing circuitry;
communication circuitry configured to communicate via a first type of communication and a second type of communication, the first type of communication being facilitated by the primary power source and the second type of communication being facilitated via an external power source; and
processing circuitry configured to:
control the communication circuitry to communicate patient data sensed via the sensing circuitry using the first type of communication when a power level of the primary power source exceeds a threshold power level;
control the communication circuitry to permanently switch from the first type of communication to the second type of communication when the power level of the primary power source falls below the threshold power level; and
control the communication circuitry to communicate patient data sensed in response to receiving power from the external power source.

2. The IMD of claim 1, wherein the processing circuitry is configured to:
control the sensing circuitry to sense patient data and to receive power from the primary power source when the power level of the primary power source exceeds the threshold power level;
control the sensing circuitry to permanently switch from being powered by the primary power source to being powered by the external power source and sensing patient data in response to receiving energy from the external power source when the power level of the primary power source falls below the threshold power level.

3. The IMD of claim 1, wherein the communication circuitry is configured to communicate via the first type of communication using a first communication component and communicate via the second type of communication using a second communication component.

4. The IMD of claim 1, wherein the first type of communication comprises Bluetooth low energy (BLE) communication.

5. The IMD of claim 1, wherein the second type of communication comprises radio frequency identification (RFID) communication.

6. The IMD of claim 1, wherein the threshold power level corresponds to expiration of the primary power source.

7. The IMD of claim 1, wherein the processing circuitry is configured to control the sensing circuitry to sense patient data according to a periodic schedule using the first type of communication when the power level of the primary power source exceeds the threshold power level.

8. The IMD of claim 1, wherein the external power source is a user device configured to receive the patient data.

9. The IMD of claim 1, wherein the IMD is injectable.

10. The IMD of claim 1, wherein the patient data comprises one or more sensed physiological signals.

11. A method comprising:
controlling, by processing circuitry of an implantable medical device (IMD), communication circuitry of the IMD to communicate patient data sensed via sensing circuitry of the IMD using a first type of communication when a power level of a primary power source of the IMD exceeds a threshold power level;
controlling, by the processing circuitry, the communication circuitry to permanently switch from the first type of communication to a second type of communication when the power level of the primary power source falls below the threshold power level; and
controlling, by the processing circuitry, the communication circuitry to communicate patient data sensed in response to receiving power from the external power source.

12. The method of claim 11, further comprising:
controlling, by the processing circuitry, the sensing circuitry to sense patient data and to receive power from the primary power source when the power level of the primary power source exceeds the threshold power level; and
controlling, by the processing circuitry, the sensing circuitry to permanently switch from being powered by the primary power source to being powered by the external power source and sensing patient data in response to receiving energy from the external power source when the power level of the primary power source falls below the threshold power level.

13. The method of claim 11, wherein the communication circuitry is configured to communicate via first type of communication using a first communication component and communicate via the second type of communication using a second communication component.

14. The method of claim 11, wherein the first type of communication comprises Bluetooth low energy (BLE) communication.

15. The method of claim 11, wherein the second type of communication comprises radio frequency identification (RFID) communication.

16. The method of claim 11, wherein the threshold power level corresponds to expiration of the primary power source.

17. The method of claim 11, wherein controlling the sensing circuitry to sense patient data using the first type of communication comprises:
controlling, by the processing circuitry, the sensing circuitry to sense patient data according to a periodic schedule using the first type of communication when the power level of the primary power source exceeds the threshold power level.

18. The method of claim 11, wherein the external power source is a user device configured to receive the patient data.

19. The method of claim 11, wherein the IMD is injectable.

20. A non-transitory computer-readable medium storing instructions that when executed cause processing circuitry to:
control communication circuitry of an implantable medical device (IMD) to communicate patient data sensed via sensing circuitry of the IMD using a first type of communication when a power level of a primary power source of the IMD exceeds a threshold power level;
control the communication circuitry to permanently switch from the first type of communication to a second type of communication when the power level of the primary power source falls below the threshold power level; and
control the communication circuitry to communicate patient data sensed in response to receiving power from the external power source.
